(19) United States
(12) Patent Application Publication
(10) Pub. No.: US 2024/0206438 A1
(43) Pub. Date: Jun. 27, 2024

(54) SIMPLE AND RAPID CONSTRUCTION OF RAT MODEL OF CONSTIPATION AND USE THEREOF

(71) Applicant: INFINITUS (CHINA) COMPANY LTD., Guangdong (CN)

(72) Inventors: Yaqian GAO, Guangdong (CN); Huiqiong ZENG, Guangdong (CN); Jian TANG, Guangdong (CN); Yunlin ZENG, Guangdong (CN); Keke HE, Guangdong (CN); Yiting YANG, Guangdong (CN); Hongying LI, Guangdong (CN); Xiaoli WEI, Guangdong (CN); Ya CAI, Guangdong (CN)

(73) Assignee: INFINITUS (CHINA) COMPANY LTD., Guangdong (CN)

(21) Appl. No.: 18/301,271

(22) Filed: Apr. 17, 2023

(30) Foreign Application Priority Data
Dec. 26, 2022 (CN) 202211676664.0

(51) Int. Cl. A01K 67/02

(52) U.S. Cl. CPC A01K 67/02; A01K 2207/10; A01K 2207/25; A01K 2207/30; A01K 2227/105; A01K 2267/03

(57) ABSTRACT

The present invention provides a simple and rapid construction of an animal model of constipation and use thereof. The construction method comprises: irritating a rat via tail-clamping and then gavaging the rat with 3.5-7 mg/kg of loperamide the next day, wherein the tail-clamping irritation treatment is as follows: clamping a tail 2-4 times every day for 25-35 min each time continuously for 3-5 days. The present invention obtains a method for constructing a rat model of constipation caused by liver depression and spleen deficiency by combining the tail-clamping irritation treatment and loperamide gavage treatment. The method has a short modeling period, is simple to operate, has a low cost, and effectively overcomes stress reaction of a rat. Besides, the constructed rat model of constipation caused by liver depression and spleen deficiency has various mechanisms, being stable, reliable and high in repeatability.

### CROSS-REFERENCE TO RELATED APPLICATION

This application claims the priority benefit of China application serial no. 202211676664.0, filed on Dec. 26, 2022. The entirety of the above-mentioned patent application is hereby incorporated by reference herein and made a part of this specification.

### TECHNICAL FIELD

The present invention belongs to the technical field of model construction and verification. More specifically, the present invention relates to a simple and rapid construction of an animal model of constipation and use thereof.

### BACKGROUND

Constipation can seriously affect life, and physical and mental health of people, reduce work and learning efficiency, bring anorectal diseases such as hemorrhoids, anal fissure, rectocele and the like. Patients with long-term drug abuse would even suffer melanosis coli, rectal mucosa relaxation, perineal descent and the like. Constipation causes feces to stay in the body, and absorption of harmful substances causes dysfunctions of gastrointestinal nerves, which in turn causes inappetence, abdominal distension, etc. Constipation may also induce onset of cardiovascular and cerebrovascular diseases, and affects brain functions, etc. The incidence rate of constipation in men is lower than that in women in China. The constipation often occurs in the middle-aged and elderly. The incidence rate of constipation in females is higher than that in males mainly due to unique physiological structure, psychological factors, diet and beauty pursuit of females. The high incidence rate of the middle-aged and the elderly is caused by hypofunction of gastrointestinal peristalsis, reduction of exercise frequency and the like.

Constipation can be classified into organic constipation and functional constipation. Organic constipation refers to constipation caused by pathological changes of organs, such as digestive tract diseases, endocrine and metabolic diseases, and requires professional medical treatment. Functional constipation refers to constipation caused by a temporary disorder of an intestinal function, and is clinically manifested as a defecation frequency less than 3 times/week, prolonged defecation interval time, difficult defecation and the like. The functional constipation mainly relates to changes of life laws, such as emotional depression, dietary habits, living habits, drug action and the like, and can be adjusted and relieved through eating functional health foods, changing living habits and the like.

Constipation caused by liver depression and spleen deficiency mainly results from emotional disorders. Intrinsic factors are abnormal regulation of colon nerves, abnormal functions of interstitial cells of Cajal (ICCs), damage of intestinal tract structural stability, slow contraction of smooth muscles and the like. The constipation caused by liver depression and spleen deficiency is a common constipation type in functional constipation. In order to relieve pains of individuals with constipation, construction of an animal model of constipation caused by liver depression and spleen deficiency is very important for screening and evaluating drugs for preventing and treating the constipation.

Tang Shuihua et al. used a combined method of tail-clamping irritation treatment and compound diphenoxylate gavage treatment to construct a rat model of qi-stagnation type constipation (Tang Shuihua, Li Sihan, Lin Xiangying, Ke Xiao, Ke Minhui, Huang Minghan, and Yang Chunbo. Effects of Liqi Tongbian Formula on brain-gut peptides in rats with functional constipation of qi stagnation pattern [J]. Journal of Beijing University of Traditional Chinese Medicine, 2021, 44 (07): 615-624). However, the method needs 14 days for modeling and consumes a long time. Moreover, the qi-stagnation type constipation mostly causes viscera-qi stagnation, abnormal unobstruction and descending, internal stasis of dregs and dry stool due to qi stagnation, qi movement disorder and the like. However, the constipation caused by liver depression and spleen deficiency is incoordinated spleen and stomach mainly caused by emotional factors and dregs in the intestines cannot be treated normally. Therefore, the rat model of qi-stagnation type constipation and the rat model of constipation caused by liver depression and spleen deficiency have obviously different etiologies and syndromes and the like, and thus the construction method cannot be used repeatedly.

### SUMMARY

Aiming at the defects of the prior art, the present invention aims to provide a simple and rapid construction of a rat model of constipation caused by liver depression and spleen deficiency, and use thereof. The present invention synthesizes various occurrence mechanisms of constipation caused by liver depression and spleen deficiency, also shortens a modeling period, and reduces a modeling cost. The operability and convenience for screening, function evaluation, mechanism research and the like of a drug, a functional food and a health-care food for preventing and treating the constipation caused by liver depression and spleen deficiency are increased, so as to provide a copying and researching tool for the constipation caused by liver depression and spleen deficiency.

A first objective of the present invention is to provide a construction method for a rat model of constipation caused by liver depression and spleen deficiency.

A second objective of the present invention is to provide use of the animal model of constipation caused by liver depression and spleen deficiency constructed by the method according to the above in screening and/or evaluating a drug for resisting constipation caused by liver depression and spleen deficiency.

The foregoing objectives of the present invention are realized by the following technical solution:

The present invention provides a construction method for a rat model of constipation caused by liver depression and spleen deficiency, comprising: irritating a rat via tail-clamping and then gavaging the rat with 3.5-7 mg/kg of loperamide the next day, wherein the tail-clamping irritation treatment is as follows: clamping a tail 2-4 times every day for 25-35 min each time continuously for 3-5 days.

Preferably, the rat is a Sprague Dawley rat (SD rat).

Further preferably, the Sprague Dawley rat has a body weight of 180-220 g.

Further preferably, the Sprague Dawley rat has a body weight of 180-200 g.

Preferably, the tail-clamping irritation treatment is as follows: clamping a tail 2-4 times every day for 30 min each time continuously for 4 days. The present invention obtains a method for constructing a rat model of constipation caused by liver depression and spleen deficiency by combining the tail-clamping irritation treatment and loperamide gavage treatment. The method has a short modeling period (only 5 days needed), is simple to operate, and has strong specificity and a low cost. Besides, the constructed rat model of constipation caused by liver depression and spleen deficiency has various mechanisms, has high stability, mainly focuses on emotional factors, and well reflects physiological characteristics of constipation caused by liver depression and spleen deficiency.

Preferably, each tail-clamping is performed at an internal of 30 min or more.

Preferably, a pathological specimen of the rat model of constipation caused by liver depression and spleen deficiency is a colon tissue of the rat.

Preferably, the tail-clamping is clamping ⅙-½ of a tail distal end of the rat.

Further preferably, the tail-clamping is clamping ⅓ of a tail distal end of the rat.

Preferably, during the tail-clamping irritation treatment, an injured part of the rat is applied with iodophor.

Preferably, during the construction process of the rat model of constipation caused by liver depression and spleen deficiency, the rat eats freely.

The rats treated by the above method have at least 7 of the following 9 symptoms: (1) the number of defecated granules is reduced within 24 hours; (2) an intestinal propulsive rate decreases; (3) a defecation weight is reduced; (4) a colon weight is decreased; (5) epithelial cells fall off or local submucosa presents edema in colon pathological tissues, connective tissues are loosely arranged, cytoplasma is loose and lightly stained, but a lamina propria is rich in intestinal glands, more goblet cells can be seen, and no obvious inflammation is seen; (6) an expression level C-Kit in Cajal cells is decreased; (7) an expression level of SCF in Cajal cells is decreased; (8) food intake is reduced; and (9) body weight is lowered. The rat model of constipation caused by liver depression and spleen deficiency can be successfully constructed by the above-mentioned method, and a screening and evaluation tool for a drug for preventing and treating constipation caused by liver depression and spleen deficiency is provided. Therefore, use of the rat model of constipation caused by liver depression and spleen deficiency constructed by the method according to the above method in screening and/or evaluating a drug for resisting constipation caused by liver depression and spleen deficiency should be within a protection range of the present invention.

The present invention has the following beneficial effects:

1. The present invention obtains a method for constructing a rat model of constipation caused by liver depression and spleen deficiency by combining the tail-clamping irritation treatment and loperamide gavage treatment. The method has a short modeling period, is simple to operate, has a low cost, and effectively overcomes stress reaction of a rat. Besides, the constructed rat model of constipation caused by liver depression and spleen deficiency has various mechanisms, is stable, reliable and high in repeatability, mainly focuses on emotional factors, and well reflects physiological characteristics of constipation caused by liver depression and spleen deficiency.

2. The model rat constructed by the method of the present invention has a higher survival rate, and a defect that the survival rate of the rat is easily reduced by an existing method (such as a water-limiting method, an acetic acid gavage method and the like) is effectively overcome. Besides, the model rat constructed by the method has no obvious changes in body weight and food intake. Technical problems that overall health of the rat is influenced and other adverse factors are brought due to an obvious reduction of body weight and/or food intake of an existing model rat thereby influencing an accuracy of drug screening and/or an evaluation result are solved by the method effectively. The method has advantages of controllable damage to the rat and objective constipation characteristics.

### BRIEF DESCRIPTION OF DRAWINGS

FIG. 1 is an observation result of a colon pathological tissue morphology of a blank group;

FIG. 2 is an observation result of a colon pathological tissue morphology of example 1;

FIG. 3 is an observation result of a colon pathological tissue morphology of example 2;

FIG. 4 is an observation result of a colon pathological tissue morphology of example 3;

FIG. 5 is an observation result of a colon pathological tissue morphology of example 4;

FIG. 6 is an observation result of a colon pathological tissue morphology of comparative example 1; and

FIG. 7 is an observation result of a colon pathological tissue morphology of comparative example 2.

### DETAILED DESCRIPTION OF EMBODIMENTS

The present invention is further described with reference to the drawings and specific examples, which are not intended to limit the present invention in any form. Unless otherwise specified, the reagents, methods and equipment used in the present invention are conventional in the art.

Unless otherwise specified, the reagents and materials used in the examples are commercially available.

Experimental animals: male SPF-grade Sprague-Dawley (SD) rats purchased from Beijing HFK Bioscience Co., Ltd with a permission number of SCXK (Jing) 2014-0004. The SD rats as experimental animals were fed under a barrier system to adapt to an environment for 5-7 days and had a body weight of 200±20 g at an end of an adaptation period.

### Example 1

### Construction Method of Rat Model of Constipation Caused by Liver Depression and Spleen Deficiency

SD rats were randomly selected, ate freely, and were subjected to timing tail-clamping irritation treatment at 9:00 and 14:00 respectively every day within the first 1-4 days. Specifically, ⅓ of a tail distal end of a rat was wrapped by sterilized gauze and clamped using a long sponge clamp, and no skin was broken. The rat was made furious and fought with other rats to irritate the rats in the whole cage, and tail-clamping was performed for 30 min each time. The rat was subjected to tail-clamping stimulation continuously within half an hour, along with aggravation of fighting, and may be scratched, and in order to avoid inflammation interference, 0.5% iodophor may be used for applying an injured part to control infection.

On day 5, the rats were gavaged with 3.5 mg/kg of loperamide without tail-clamping treatment.

### Example 2

### Construction Method of Rat Model Of Constipation Caused by Liver Depression and Spleen Deficiency

The method was the same as that in example 1 only in a difference that a dosage of the loperamide gavaged on day 5 was 7 mg/kg.

### Example 3

### Construction Method of Rat Model of Constipation Caused by Liver Depression and Spleen Deficiency

The method was the same as that in example 1 only in a difference that the tail-clamping was performed 4 times a day at 9:00, 14:00, 17:00 and 21:00 respectively every day.

### Example 4

### Construction Method of Rat Model of Constipation Caused by Liver Depression and Spleen Deficiency

The method was the same as that in example 3 only in a difference that a dosage of the loperamide gavaged on day 5 was 7 mg/kg.

### Comparative Example 1

SD rats were randomly selected, ate freely, and gavaged with 3.5 mg/kg of loperamide every day without tail-clamping treatment within 1-7 days.

### Comparative Example 2

SD rats were randomly selected, ate freely, and gavaged with 3.5 mg/kg of loperamide every day without tail-clamping treatment within 1-14 days.

### Application Example

The SD rats of examples 1-4 and comparative examples 1-2 were selected and SD rats without any treatment (ate freely for 14 days only) were as a blank group. The rats were subjected to the following evaluation tests.

### I. Evaluations on Main Symptoms

### 1. Evaluation on Symptoms During Test

(1) Measurement of number of defecated granules and defecation weight within 24 hours: during the test, feces of each SD rat within 24 hours in a metabolism cage were collected at 10:00 a.m. every day, the number of defecated granules was observed and the defecation weight was measured.

(2) Measurement of body weight change: during the test, the body weight of each SD rat was measured at 9:00 a.m. every day, each rat was measured three times, and an average value was taken.

(3) Measurement of food intake change: during the test, food intake of each SD rat was recorded at 20:00 p.m. every day.

### 2. Evaluation on Symptoms After Completion of Test

After the test (after a last gavage of loperamide) was ended, no death of the SD rats was observed. Each SD rat was fasted for 12 hours without water deprivation and each group was randomly divided into two sub-groups, i.e., a first sub-group and a second sub-group with 5 rats in each. Then, the rats were subjected to the following tests:

### (1) Measurement of Intestinal Propulsive Rate and Colon Weight

The SD rats in the first sub-group were respectively gavaged with 1 mL/100 g of homemade ink (100 g of Arabic gum was accurately weighed, 800 mL of distilled water was added, the mixture was heated and boiled until a solution was transparent, then 50 g of activated carbon powder was added, the solution was continuously boiled for 3 times, after the solution was cooled to 25° C., distilled water was added to set a constant volume of 1,000 mL, and the solution was stored in a refrigerator at 4° C. and shaken uniformly before use). The rats were sacrificed 30 min after the gavage, the abdomen was cut to take out all gastrointestinal tracts, the weight of colon was weighed, and a distance (cm) from a front end of the ink to the pyloric sphincter and a distance (cm) from the pyloric sphincter to a tail end of a small intestine were measured. An intestinal propulsive rate was calculated according to a formula “intestinal propulsive rate =distance (cm) from front end of ink to pyloric sphincter/distance (cm) from pyloric sphincter to tail end of small intestine×100%”.

### (2) Observation of Colon Pathological Tissue Morphology And Measurement Of C-Kit and SCF Expression Levels in Cajal Cells

The SD rats in the second sub-group were sacrificed, colon tissue samples (a part about 2 cm away from the cecum and a colon tissue was reserved) were taken. The mRNA expression levels of c-Kit, SCF and internal reference U6 in the Cajal cells (the expression level of the internal reference U6 was stable in most cases and usually used as the internal reference) were detected by using a RT-PCR technology. The relative expression levels of the c-Kit and SCF were obtained by converting the expression levels of the c-Kit, SCF with the internal reference U6. Then the samples were fixed with 10% (v/v) formaldehyde for 24 hours and subjected to HE staining. Pathological changes of a mucosa layer, a lamina propria and a muscular layer and inflammation were observed by an optical microscope.

### II. Statistical Method

Experiment data were analyzed by One-way ANOVA in an analysis of variance and the results were expressed as mean±standard deviation (X±SD).

III. Experimental Results 1. The results of the number of defecated granules within 24 hours were shown in Table 1.

TABLE 1
Number of defecated granules within 24 hours in each group of rats (unit: granules)

| | Blank | | | | | Comparative | Comparative |
|---|---|---|---|---|---|---|---|
| Days | group | Example 1 | Example 2 | Example 3 | Example 4 | example 1 | example 2 |
| 0 | 47.72 ± 1.02 | 45.14 ± 1.59 | 46.67 ± 2.35 | 46.79 ± 1.54 | 45.53 ± 2.05 | 46.34 ± 2.40 | 45.30 ± 2.74 |
| 1 | 49.62 ± 1.72 | 41.05 ± 2.52** | 39.64 ± 2.19** | 40.24 ± 1.57** | 41.14 ± 1.95** | 44.87 ± 4.66* | 43.32 ± 4.22* |
| 2 | 48.00 ± 1.90 | 40.71 ± 1.42** | 37.94 ± 3.05** | 39.10 ± 1.61** | 40.75 ± 1.96** | 45.43 ± 0.27* | 44.91 ± 2.25* |
| 3 | 47.17 ± 2.93 | 40.55 ± 2.15** | 35.79 ± 2.04** | 38.49 ± 2.21** | 39.76 ± 2.02** | 46.77 ± 1.82 | 47.29 ± 2.14 |
| 4 | 46.16 ± 2.37 | 35.11 ± 1.65** | 32.12 ± 3.27** | 46.91 ± 1.92** | 37.67 ± 1.51** | 45.58 ± 1.76 | 46.17 ± 1.50 |
| 5 | 45.50 ± 2.43 | 30.89 ± 2.75** | 24.85 ± 2.23** | 35.45 ± 2.01** | 36.45 ± 2.07** | 46.92 ± 2.45 | 47.32 ± 1.41 |
| 6 | 47.22 ± 1.52 | — | — | — | — | 46.42 ± 3.12 | 45.42 ± 1.86 |
| 7 | 46.45 ± 2.40 | — | — | — | — | 45.64 ± 1.51 | 46.38 ± 2.04 |
| 8 | 47.53 ± 3.26 | — | — | — | — | — | 45.27 ± 1.49 |
| 9 | 49.12 ± 1.64 | — | — | — | — | — | 47.33 ± 1.34 |
| 10 | 48.67 ± 1.04 | — | — | — | — | — | 47.96 ± 2.54 |
| 11 | 46.25 ± 2.73 | — | — | — | — | — | 45.64 ± 2.20 |
| 12 | 45.81 ± 1.21 | — | — | — | — | — | 46.99 ± 1.01 |
| 13 | 47.65 ± 2.45 | — | — | — | — | — | 45.85 ± 2.74 |
| 14 | 50.74 ± 3.64 | — | — | — | — | — | 46.19 ± 2.10 |

Note:
compared with the blank group, *p < 0.05 and **p < 0.01.

It can be seen that:

(1) Compared with the blank group, within 1-5 days, the number of defecated granules within 24 hours in examples 1-4 was remarkably decreased with a statistical significance (all p<0.01), wherein the number of defecated granules in example 2 was the lowest. The results indicated that the present invention used the tail-clamping irritation treatment and loperamide gavage treatment under a specific condition, which can obviously reduce the number of feces granules and well simulated the characteristics of constipation caused by liver depression and spleen deficiency.

(2) The effects of examples 1-2 with 2 tail-clampings every day were better than those of examples 3-4 with 4 tail-clampings every day, indicating that more tail-clamping times did not have a better effect. Through unremitting exploration by the present invention, it was found that the tail-clamping treatment as an emotional stimulation means was too severe, the rat would generate stress reaction, thereby counteracting an influence caused by the tail-clamping to different degrees.

(3) The number of defecated granules within 24 hours in comparative examples 1-2 had no significant difference compared with the blank group in a later period. The present invention found that along with prolonging intake time of loperamide, the SD rat had an extremely high adaptability change capability and showed a self-repairing phenomenon due to the existing stress reaction. Therefore, a constipation model constructed by taking 7 days or 14 days as a gavage period of the loperamide had lower reliability.

2. The results of the intestinal propulsive rates were shown in Tables 2.

TABLE 2
Intestinal propulsive rates of rats in each group (unit: %)

| | Propulsive rate |
|---|---|
| Blank group | 56.04 ± 0.96 |
| Example 1 | 35.61 ± 2.14** |
| Example 2 | 30.33 ± 1.55** |
| Example 3 | 41.54 ± 2.62** |
| Example 4 | 42.25 ± 1.72** |
| Comparative example 1 | 55.34 ± 2.75 |
| Comparative example 2 | 54.59 ± 3.15 |

Note:
compared with the blank group, *p < 0.05 and **p < 0.01.

It can be seen that:

(1) The intestinal propulsive rate of examples 1-4 was all significantly lower than that of the blank group with a significant difference (all p<0.01), wherein the intestinal propulsive rate of example 2 was the lowest. The results indicated that the present invention used the tail-clamping irritation treatment and loperamide gavage treatment under a specific condition, which can obviously reduce an intestinal peristalsis speed and had an obvious effect on simulating a characteristic of slowing down an intestinal movement by constipation caused by liver depression and spleen deficiency.

(2) The intestinal propulsive rate in comparative examples 1-2 had no significant difference compared with the blank group. The present invention found that along with prolonging intake time of loperamide, the SD rat had an extremely high adaptability change capability and showed a self-repairing phenomenon due to the existing stress reaction. Therefore, a constipation model constructed by taking 7 days or 14 days as a gavage period of the loperamide had lower reliability.

3. The results of defecation weight within 24 hours were shown in Table 3.

TABLE 3
Defecation weight within 24 hours in each group of rats (unit: g)

| | Blank | | | | | Comparative | Comparative |
|---|---|---|---|---|---|---|---|
| Days | group | Example 1 | Example 2 | Example 3 | Example 4 | example 1 | example 2 |
| 0 | 9.85 ± 0.17 | 10.12 ± 0.41 | 9.45 ± 0.21 | 10.21 ± 0.37 | 10.05 ± 0.44 | 9.94 ± 0.53 | 10.07 ± 0.45 |
| 1 | 9.17 ± 0.26 | 6.95 ± 0.15** | 6.74 ± 0.05** | 7.01 ± 0.20** | 7.95 ± 0.32** | 6.19 ± 0.20** | 6.50 ± 0.14** |
| 2 | 10.50 ± 0.32 | 6.35 ± 0.27** | 6.50 ± 0.15** | 7.27 ± 0.29** | 7.41 ± 0.19** | 7.44 ± 2.57* | 6.70 ± 2.33* |
| 3 | 10.25 ± 0.34 | 6.43 ± 0.16** | 6.00 ± 0.20** | 6.72 ± 0.31** | 6.91 ± 0.24** | 9.19 ± 1.50 | 9.50 ± 0.94 |
| 4 | 10.09 ± 0.38 | 6.04 ± 0.22** | 5.33 ± 0.10** | 6.55 ± 0.16** | 5.94 ± 0.12** | 9.74 ± 2.65 | 10.96 ± 1.07 |
| 5 | 10.54 ± 0.60 | 5.56 ± 0.30** | 5.15 ± 0.42** | 6.09 ± 0.04** | 5.62 ± 0.07** | 9.31 ± 2.77 | 10.11 ± 0.40 |
| 6 | 11.65 ± 0.57 | — | — | — | — | 10.05 ± 1.09 | 10.47 ± 0.35 |
| 7 | 11.72 ± 0.46 | — | — | — | — | 11.81 ± 2.12 | 10.39 ± 0.54 |
| 8 | 11.85 ± 0.71 | — | — | — | — | — | 11.49 ± 1.23 |
| 9 | 11.29 ± 0.55 | — | — | — | — | — | 11.62 ± 1.04 |
| 10 | 11.52 ± 0.72 | — | — | — | — | — | 11.49 ± 1.76 |
| 11 | 11.79 ± 0.54 | — | — | — | — | — | 11.54 ± 0.28 |
| 12 | 12.57 ± 0.49 | — | — | — | — | — | 11.88 ± 0.45 |
| 13 | 12.76 ± 0.73 | — | — | — | — | — | 12.46 ± 0.19 |
| 14 | 12.89 ± 0.47 | — | — | — | — | — | 12.62 ± 0.51 |

Note:
compared with the blank group, **p < 0.01.

It can be seen that:

(1) Compared with the blank group, within 1-5 days, the defecation weight within 24 hours in examples 1-4 was remarkably decreased with a statistical significance (all p <0.01), wherein the defecation weight in example 2 was the lowest. The results indicated that the present invention used the tail-clamping irritation treatment and loperamide gavage treatment under a specific condition, which can obviously reduce the defecation weight, influenced an intestinal transportation, and had an obvious effect on simulating a characteristic of slowing down the intestinal transportation by constipation caused by liver depression and spleen deficiency.

(2) The defecation weight within 24 hours in comparative examples 1-2 had no significant difference compared with the blank group in a later period. The present invention found that along with prolonging intake time of loperamide, the SD rat had an extremely high adaptability change capability and showed a self-repairing phenomenon due to the existing stress reaction. Therefore, a constipation model constructed by taking 7 days or 14 days as a gavage period of the loperamide had lower reliability.

4. The measurement of colon weight was shown in Table 4.

TABLE 4
Colon weight in each group of rats (unit: g)

| | Colon weight |
|---|---|
| Blank group | 1.60 ± 0.10 |
| Example 1 | 2.43 ± 0.06** |
| Example 2 | 2.52 ± 0.14** |
| Example 3 | 2.27 ± 0.25** |
| Example 4 | 2.37 ± 0.17** |
| Comparative example 1 | 1.77 ± 0.32 |
| Comparative example 2 | 1.68 ± 0.24 |

Note:
compared with the blank group, *p < 0.05 and **p < 0.01.

The colon weight selected by the test comprises feces and tissue weight of the whole colon section, and reflects the residual amount of feces and a strength of an intestinal peristalsis function. It can be seen that compared with the blank group, the colon weight of examples 1-4 was obviously higher than that of the blank group with a significant difference (all p<0.01). The results indicated that the present invention used the tail-clamping irritation treatment and loperamide gavage treatment under a specific condition, can prolong transportation time of an intestinal content in the colon and had an obvious effect on simulating a characteristic of slowing down a feces transportation by constipation caused by liver depression and spleen deficiency.

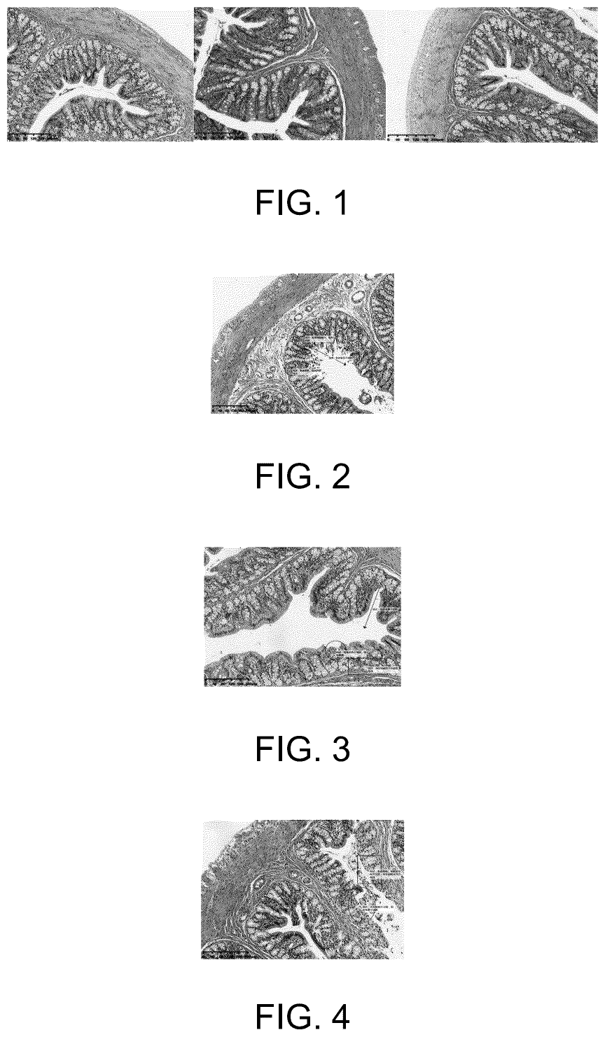

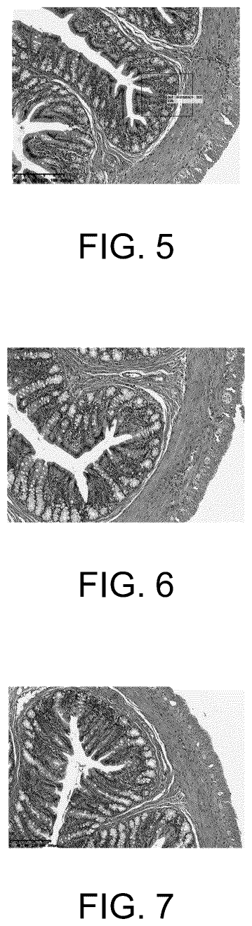

5. The observation results of colon pathological tissue morphologies were shown in FIGS. 1-5, wherein FIG. 1 was the observation result of a colon pathological tissue morphology of a blank group; FIG. 2 was the observation result of a colon pathological tissue morphology of example 1; FIG. 3 was the observation result of a colon pathological tissue morphology of example 2; FIG. 4 was the observation result of a colon pathological tissue morphology of example 3; FIG. 5 was the observation result of a colon pathological tissue morphology of example 4; FIG. 6 was the observation result of a colon pathological tissue morphology of comparative example 1; and FIG. 7 was the observation result of a colon pathological tissue morphology of comparative example 2.

It can be seen that after the modeling was ended, gland structures of colon mucosa epithelium, a lamina propria, a muscular layer and a submucosa of the rats in the blank group were clear and regular and completely arranged, a surface of a crypt was regular and intact, a shape of the crypt was complete, and the crypts were orderly arranged; and goblet cells were tightly connected without a disorder phenomenon, smooth muscle cells were normally distributed, obvious inflammation was not seen, and comparative examples 1-2 were similar to the blank group. Glandular structures of colon mucosa epithelium, a lamina propria, a muscular layer and a submucosa of the rats in example 1-5 groups were clear and regular, but goblet cells were incomplete and not arranged closely and orderly, a surface of a crypt was irregular, the number of crypts was reduced, and the crypts shrunk; and smooth muscle cells were normally distributed and no obvious inflammation was seen. The results indicated that the present invention used the tail-clamping irritation treatment and loperamide gavage treatment under a specific condition, which well simulated a characteristic of a colon tissue in the constipation caused by liver depression and spleen deficiency.

6. The measurement results of relative expression levels of C-Kit in Cajal cells were shown in Table 5.

TABLE 5
Relative expression levels of C-Kit in
colon Cajal cells in each group of rats

| | Relative expression level of C-Kit |
|---|---|
| Blank group | 2.64 ± 0.10 |
| Example 1 | 1.51 ± 0.05** |
| Example 2 | 1.24 ± 0.03** |
| Example 3 | 1.74 ± 0.11** |
| Example 4 | 1.85 ± 0.21** |
| Comparative example 1 | 2.54 ± 0.26 |
| Comparative example 2 | 2.49 ± 0.17 |

Note:
compared with the blank group, *p < 0.05 and **p < 0.01.

As can be seen, the relative expression level of C-Kit in the Cajal cells in examples 1-4 was all obviously lower than that in the blank group with a significant difference (all p<0.01), wherein the expression level of C-Kit was the lowest in example 2. The results indicated that the present invention used the tail-clamping irritation treatment and loperamide gavage treatment under a specific condition, which can obviously decrease the expression level of C-Kit in the Cajal cells, damage interstitial cells of Cajal of the colon, slow down an intestinal peristalsis, and enable a characteristic of the constipation caused by liver depression and spleen deficiency to be obvious.

7. The measurement results of relative expression levels of SCF in Cajal cells were shown in Table 6.

TABLE 6
Relative expression levels of SCF in colon
Cajal cells in each group of rats

| | Relative expression level of SCF |
|---|---|
| Blank group | 1.44 ± 0.06 |
| Example 1 | 0.95 ± 0.07** |
| Example 2 | 0.88 ± 0.02** |
| Example 3 | 1.13 ± 0.05** |
| Example 4 | 1.12 ± 0.04** |
| Comparative example 1 | 1.40 ± 0.12 |
| Comparative example 2 | 1.49 ± 0.09 |

Note:
compared with the blank group, *p < 0.05 and **p < 0.01.

As can be seen, the relative expression level of SCF in the Cajal cells in examples 1-4 was all obviously lower than that in the blank group with a significant difference (all p<0.01), wherein the expression level of SCF was the lowest in example 2. Since the interstitial cells of Cajal can survive only under a certain concentration of SCF, the reduction of the SCF expression level would reduce the number of the interstitial cells of Cajal, further reduce the expression level of C-Kit of the interstitial cells of Cajal, damage the colon interstitial cells of Cajal and slow down an intestinal peristalsis. The results indicated that examples 1 -4 can simulate a deep mechanism of the constipation caused by liver depression and spleen deficiency.

8. The measurement results of body weight gain changes were shown in Table 7.

TABLE 7
Body weight change of rats in each group (unit: g)

| | | | | | | Comparative | Comparative |
|---|---|---|---|---|---|---|---|
| Days | Blank group | Example 1 | Example 2 | Example 3 | Example 4 | example 1 | example 2 |
| 0 | 212.54 ± 5.05 | 215.44 ± 5.31 | 214.87 ± 5.49 | 209.19 ± 2.97 | 207.72 ± 5.32 | 211.45 ± 4.77 | 210.02 ± 3.56 |
| 1 | 227.72 ± 5.40 | 224.89 ± 4.56 | 225.65 ± 6.95 | 221.54 ± 4.32 | 219.69 ± 2.68 | 210.78 ± 6.19 | 213.43 ± 2.74 |
| 2 | 252.16 ± 7.21 | 245.75 ± 2.54 | 247.67 ± 4.93 | 242.26 ± 6.24 | 241.79 ± 5.61 | 223.52 ± 4.07 | 224.07 ± 7.57 |
| 3 | 274.59 ± 4.67 | 270.25 ± 4.60 | 265.34 ± 5.76 | 267.72 ± 2.96 | 265.78 ± 6.27 | 242.09 ± 7.50 | 241.92 ± 6.14 |
| 4 | 303.42 ± 5.35 | 297.15 ± 7.53 | 295.62 ± 4.52 | 297.52 ± 3.70 | 298.01 ± 4.21 | 264.14 ± 8.15 | 267.85 ± 6.88 |
| 5 | 324.94 ± 2.05 | 317.97 ± 3.65 | 319.74 ± 5.41 | 315.62 ± 7.54 | 312.95 ± 5.64 | 296.44 ± 7.34 | 294.30 ± 7.47 |
| 6 | 332.47 ± 4.90 | — | — | — | — | 321.02 ± 7.14 | 316.86 ± 5.99 |
| 7 | 337.56 ± 2.12 | — | — | — | — | — | 327.01 ± 6.11 |
| 8 | 342.85 ± 5.20 | — | — | — | — | — | 331.32 ± 8.51 |
| 9 | 349.04 ± 3.75 | — | — | — | — | — | 335.70 ± 6.82 |
| 10 | 353.55 ± 4.17 | — | — | — | — | — | 345.29 ± 7.18 |
| 11 | 358.81 ± 2.32 | — | — | — | — | — | 351.32 ± 6.74 |
| 12 | 362.72 ± 5.47 | — | — | — | — | — | 353.22 ± 5.09 |
| 13 | 367.06 ± 5.71 | — | — | — | — | — | 360.92 ± 4.50 |
| 14 | 369.24 ± 5.07 | — | — | — | — | — | 365.40 ± 6.15 |

Note:
compared with the blank group, *p < 0.05 and **p < 0.01.

It can be seen that after the modeling was ended, the body weight of the rats in examples 1-4 was slightly reduced compared with that of the blank group without a statistical significance, indicating the modeling method of the present invention had no significant influence on the body weight of the rats. The method effectively solves technical problems that overall health of the rat was influenced and other adverse factors were brought due to an obvious reduction of body weight of an existing model rat and thus an accuracy of drug screening and/or an evaluation result was influenced.

9. The measurement results of food intake were shown in Table 8.

TABLE 8
Food intake of rats in each group (unit: g)

| | Blank | | | | | Comparative | Comparative |
|---|---|---|---|---|---|---|---|
| Days | group | Example 1 | Example 2 | Example 3 | Example 4 | example 1 | example 2 |
| 0 | 16.24 ± 1.06 | 14.16 ± 1.74 | 15.77 ± 0.85 | 15.02 ± 0.71 | 15.40 ± 0.34 | 15.21 ± 1.45 | 16.02 ± 1.22 |
| 1 | 17.32 ± 0.45 | 15.43 ± 2.65 | 16.55 ± 2.07 | 15.81 ± 1.53 | 16.29 ± 1.79 | 14.09 ± 1.06 | 15.50 ± 1.12 |
| 2 | 19.51 ± 1.62 | 17.20 ± 2.26 | 17.91 ± 2.90 | 16.79 ± 3.15 | 17.66 ± 2.60 | 15.89 ± 1.81 | 16.28 ± 2.55 |
| 3 | 20.17 ± 2.10 | 18.54 ± 2.77 | 18.74 ± 1.58 | 17.36 ± 2.31 | 18.02 ± 3.57 | 16.58 ± 3.52 | 17.65 ± 2.41 |
| 4 | 22.21 ± 1.73 | 19.85 ± 3.25 | 20.92 ± 2.61 | 19.70 ± 2.67 | 20.41 ± 2.26 | 18.82 ± 2.64 | 19.44 ± 2.10 |
| 5 | 23.34 ± 1.59 | 21.42 ± 2.80 | 22.41 ± 1.65 | 21.37 ± 2.13 | 21.54 ± 1.08 | 19.14 ± 3.01 | 20.71 ± 2.19 |
| 6 | 24.27 ± 0.29 | — | — | — | — | 20.03 ± 2.20 | 21.11 ± 0.58 |
| 7 | 24.46 ± 1.26 | — | — | — | — | 22.73 ± 1.50 | 23.88 ± 2.11 |
| 8 | 25.52 ± 1.44 | — | — | — | — | — | 24.50 ± 2.31 |
| 9 | 25.37 ± 0.92 | — | — | — | — | — | 24.35 ± 1.58 |
| 10 | 26.04 ± 1.33 | — | — | — | — | — | 25.77 ± 2.40 |
| 11 | 27.44 ± 1.42 | — | — | — | — | — | 26.10 ± 2.14 |
| 12 | 28.30 ± 2.07 | — | — | — | — | — | 27.23 ± 1.25 |
| 13 | 29.59 ± 2.25 | — | — | — | — | — | 28.05 ± 2.64 |
| 14 | 30.12 ± 1.19 | — | — | — | — | — | 29.09 ± 1.75 |

It can be seen that after the modeling was ended, the food intake of the rats in examples 1-4 was slightly reduced compared with that of the blank group without a statistical significance, indicating the modeling method of the present invention had no significant influence on the food intake of the rats. The method effectively solves technical problems that overall health of the rat was influenced and other adverse factors were brought due to an obvious reduction of food intake of an existing model rat and thus an accuracy of drug screening and/or an evaluation result was influenced.

In summary, the rats of examples 1-4 had at least the first 7 of the following 9 symptoms: (1) the number of defecated granules was reduced within 24 hours; (2) an intestinal propulsive rate decreased; (3) a defecation weight was reduced; (4) a colon weight was decreased; (5) epithelial cells fell off or local submucosa presented edema in colon pathological tissues, connective tissues were loosely arranged, cytoplasma was loose and lightly stained, but a lamina propria was rich in intestinal glands, more goblet cells can be seen, and no obvious inflammation was seen; (6) an expression level C-Kit in Cajal cells was decreased; (7) an expression level of SCF in Cajal cells was decreased; (8) food intake was reduced; and (9) body weight was lowered. The results indicated that the tail-clamping irritation treatment and loperamide gavage treatment under a specific condition can successfully construct a rat model of constipation caused by liver depression and spleen deficiency. The constructed rat model had various mechanisms, mainly focused on emotional factors, and well reflected physiological characteristics of the constipation caused by liver depression and spleen deficiency.

The above examples are preferred embodiments of the present invention. However, the embodiments of the present invention are not limited by the above examples. Any change, modification, substitution, combination and simplification made without departing from the spiritual essence and principle of the present invention should be an equivalent replacement manner, and all are included in a protection scope of the present invention.

What is claimed is:

1. A construction method for a rat model of constipation caused by liver depression and spleen deficiency, comprising: irritating a rat via a tail-clamping irritation treatment and then gavaging the rat with 3.5-7 mg/kg of loperamide the next day, wherein the tail-clamping irritation treatment is as follows: clamping a tail 2-4 times every day for 25-35 min each time continuously for 3-5 days.

2. The construction method according to claim 1, wherein the rat is a Sprague Dawley rat.

3. The construction method according to claim 1, wherein the tail-clamping irritation treatment is as follows: clamping a tail 2-4 times every day for 30 min each time continuously for 4 days.

4. The construction method according to claim 1, wherein each tail-clamping is performed at an internal of 30 min or more.

5. The construction method according to claim 1, wherein a pathological specimen of the rat model of constipation caused by liver depression and spleen deficiency is a colon tissue of the rat.

6. The construction method according to claim 1, wherein clamping the tail is clamping ⅙-½ of a tail distal end of the rat.

7. The construction method according to claim 6, wherein clamping the tail is clamping ⅓ of a tail distal end of the rat.

8. The construction method according to claim 1, wherein during the tail-clamping irritation treatment, an injured part of the rat is applied with iodophor.

9. The construction method according to claim 1, wherein during a construction process of the rat model of constipation caused by liver depression and spleen deficiency, the rat eats freely.

10. Use of the rat model of constipation caused by liver depression and spleen deficiency constructed by the method according to claim 1 in screening and/or evaluating a drug for resisting constipation caused by liver depression and spleen deficiency.

11. Use of the rat model of constipation caused by liver depression and spleen deficiency constructed by the method according to claim 2 in screening and/or evaluating a drug for resisting constipation caused by liver depression and spleen deficiency.

12. Use of the rat model of constipation caused by liver depression and spleen deficiency constructed by the method according to claim 3 in screening and/or evaluating a drug for resisting constipation caused by liver depression and spleen deficiency.

13. Use of the rat model of constipation caused by liver depression and spleen deficiency constructed by the method according to claim 4 in screening and/or evaluating a drug for resisting constipation caused by liver depression and spleen deficiency.

14. Use of the rat model of constipation caused by liver depression and spleen deficiency constructed by the method according to claim 5 in screening and/or evaluating a drug for resisting constipation caused by liver depression and spleen deficiency.

15. Use of the rat model of constipation caused by liver depression and spleen deficiency constructed by the method according to claim 6 in screening and/or evaluating a drug for resisting constipation caused by liver depression and spleen deficiency.

16. Use of the rat model of constipation caused by liver depression and spleen deficiency constructed by the method according to claim 7 in screening and/or evaluating a drug for resisting constipation caused by liver depression and spleen deficiency.

17. Use of the rat model of constipation caused by liver depression and spleen deficiency constructed by the method according to claim 8 in screening and/or evaluating a drug for resisting constipation caused by liver depression and spleen deficiency.

18. Use of the rat model of constipation caused by liver depression and spleen deficiency constructed by the method according to claim 9 in screening and/or evaluating a drug for resisting constipation caused by liver depression and spleen deficiency.
